(19) United States
(12) Patent Application Publication
(10) Pub. No.: US 2020/0113854 A1
(43) Pub. Date: Apr. 16, 2020

(54) Composition for Improving Brain Function

(71) Applicant: MORINAGA MILK INDUSTRY CO., LTD., Tokyo (JP)

(72) Inventors: Youdai Kobayashi, Kanagawa (JP); Tetsuya Kuhara, Kanagawa (JP)

(73) Assignee: MORINAGA MILK INDUSTRY CO., LTD., Tokyo (JP)

(21) Appl. No.: 16/615,485

(22) PCT Filed: May 17, 2018
(86) PCT No.: PCT/JP2018/019108

§ 371 Date: Nov. 21, 2019

(30) Foreign Application Priority Data
May 30, 2017 (JP) 2017-106200

(51) Int. Cl. A61K 31/19; A61K 9/00; A61P 25/28; A23L 33/10; A23L 2/68

(52) U.S. Cl. CPC A61K 31/19; A61K 9/0056; A23V 2002/00; A23L 33/10; A23L 2/68; A61P 25/28

(57) ABSTRACT

A composition for improving brain function contains acetic acid and/or an acetic acid salt as an active ingredient. Further suitably, the brain function improvement is brain dysfunction improvement or cognitive dysfunction improvement. Further suitably, the cognitive dysfunction is Alzheimer's-type dementia or vascular dementia. Further suitably, the composition is for oral intake. Further suitably, the composition is a food or drink composition for improving brain function.

### TECHNICAL FIELD

The present technique relates to a composition for improving brain function, for example, a brain function improver and a food or drink composition for improving brain function.

### BACKGROUND ART

Since all persons of young, middle, or old ages have always expected improvement of brain functions regardless of his/her age or gender, pharmaceuticals, herbal medicines, Chinese medicines, food or drink products, and the like, that are safe and that can be administered for a long period of time, are required.

In particular, with an extended life-span in the coming super-aging society, it is one important social issue to prevent declines in brain functions due to aging or declines in brain functions due to age-related brain diseases to extend the healthy life-span where a human can aggressively act and to suppress a decrease in quality of life (QOL) in his/her lifetime.

Furthermore, prophylaxis, treatment, or improvement of dementia in which declines in learning and memory abilities, among declines in brain functions, are gradually shown and which considerably affects daily life, is an urgent task. Besides, a decline in brain function may occur by brain damage due to a traffic accident, a cerebrovascular disorder, or the like, and symptoms from such causes are called higher brain dysfunction.

Dementia can be roughly divided into vascular dementia and Alzheimer's-type dementia.

Vascular dementia is dementia caused by necrotic nerve cells due to a cerebrovascular disorder, such as brain infarction or subarachnoid hemorrhage.

Alzheimer's-type dementia includes early-onset dementia and age-related dementia. Declines in cognitive functions and an alteration in personality are main symptoms of Alzheimer's-type dementia. Alzheimer's disease is categorized in neurodegenerative diseases which also includes Parkinson's disease and amyotrophic lateral sclerosis (ALS) (NPL 1). The cause of Alzheimer's disease has long been studied and a social need has recently been increasing for etiology and therapeutics of the disease against the background of the frequency thereof.

Alzheimer's disease accounts for 50 to 60% of nerve diseases, and the symptoms are considered to be caused by a protein, β-amyloid, accumulating in the brain. The accumulating β-amyloid breaks normal nerve cells to further cause brain atrophy (NPL 2). In addition, it is reported that the concentration of lactic acid increases in the cerebrospinal fluid of a patient of Alzheimer's disease (NPL 3).

Possible causes of Alzheimer's disease include an age-related change of the brain, a genetic factor, an environmental factor, and a lifestyle-related factor, but the causes have yet to be fully elucidated.

As a composition for improving brain function, for example, PTL 1 disclose a composition for improving brain function that contains as an active ingredient an extract mainly containing a polyphenol and obtained by extracting leaves of Ligustrum purpurascens Y. C. Yang with water, an organic solvent alone, or a mixture thereof, the extract having an antioxidative activity and/or aldose reductase inhibitory activity.

However, there have yet been few findings about a compound or the like that improves a brain function and thus further exploration and research are being promoted about compounds having a brain function improving effect.

### CITATION LIST

### Patent Literature

PTL 1: JP-A-2010-163460

### Non-Patent Literature

NPL 1: Igaku no Ayumi (Journal of Clinical and Experimental Medicine), Vol. 247, No. 5, 465-471, 2013.
NPL 2: Selkoe, D. J., Physiological Reviews, 81, 741-766, 2001
NPL 3: Liguori et al., Journal of Neurological and Neurosurgical Psychiatry, 86(6), 655-659, 2015
NPL 4: Nippon Eiyo Syokuryo Gakukaishi (Journal of Japan Society of Nutrition and Food Science), Vol. 67, No. 4, 171-176, 2014.
NPL 5: Furusawa et al., Nature 504, 446-450, 2013
NPL 6: Takeda et al., Brain Research, 1280, 137-147, 2009

### SUMMARY

In view of the above situation, a primary object of the present technique is to provide a composition for improving brain function (for example, a brain function improver or a food or drink composition for improving brain function).

Then, as a result of extensive studies, the present inventors surprisingly found that when acetic acid and/or an acetic acid salt was orally taken in a cognitive function determination model, decline in cognitive function was able to be suppressed. Based on the result, the present inventors have found that acetic acid and/or an acetic acid salt, which is safe, can improve brain function.

NPL 4 discloses that acetic acid is used as a living body fuel, that acetic acid activates AMP kinase, and that acetic acid has an obesity suppressing action and a lipid metabolism promoting action. However, direct relationship between acetic acid and brain function improvement is not known.

In addition, NPL 5 discloses the induction of regulatory T-cell differentiation by butyric acid produced by enteric bacteria, but states that no induction of regulatory T-cell differentiation was found by acetic acid. Thus, probiotic actions have very complicated mechanisms. Accordingly, compounds having similar structures do not always serve the same function in the living body (particular in oral intake).

There has yet been no report of brain function improvement by taking acetic acid.

Specifically, the present technique includes a composition for improving brain function (for example, brain function improver or food or drink composition for improving brain function) containing acetic acid and/or an acetic acid salt as an active ingredient.

The improving brain function may be improving brain dysfunction.

The improving brain function may be improving cognitive dysfunction.

The cognitive dysfunction may be Alzheimer's-type dementia or vascular dementia.

The composition for improving brain function may be for oral intake.

The improving brain function mentioned in the present technique means improvement particularly in a cognitive function (for example, memory, thinking, language, determination, calculation, affect, or the like). Examples of declines in brain functions include a brain function decline by aging, brain dysfunction due to an age-related brain disease, and brain dysfunction due to brain damage.

Examples of symptoms of brain dysfunction include higher brain dysfunction, vascular dementia, and Alzheimer's-type dementia.

Alzheimer's-type dementia includes early-onset dementia and dementia by aging.

Note that acetic acid and/or an acetic acid salt in the present technique may be used for a human or a non-human animal (suitably mammal) as a subject, preferably for a human and a pet, more preferably for a human. The technique may be used for a therapeutic purpose or for a nontherapeutic purpose.

“Nontherapeutic purpose” has a concept not including any medical practice, that is, therapeutic treatment practices on a human body. Examples thereof include beauty actions and health promotion.

“Improvement” means change for the better of a disease, a symptom, or a condition; prevention or delaying of worsening of a disease, a symptom, or a condition; and reversing, prevention, or delaying of progression of a disease or a symptom.

“Prophylaxis” means prevention or delaying of onset of a disease or a symptom in a subject, or reduction in the risk of a disease or a symptom of a subject.

### Advantageous Effects

The present technique can provide a composition for improving brain function (for example, brain function improver or food or drink composition for improving brain function).

Note that the effect is not always limited to those described above and any effect described in the technique may be applied.

### DETAILED DESCRIPTION

A suitable embodiment for implementing the present technique will be described below. Note that the embodiment described below is an example of a typical embodiment of the present technique and the scope of the present technique is not to be narrowly construed by the embodiment.

The present technique includes a composition for improving brain function containing acetic acid and/or an acetic acid salt as an active ingredient. The composition for improving brain function includes a brain function improver, a pharmaceutical composition for improving brain function, a food or drink composition for improving brain function, and the like. Among them, an oral composition for improving brain function (for example, an oral brain function improver and a food or drink composition for improving brain function) is preferred since efficacy can be exhibited in oral intake as shown in Examples described later herein. In oral intake, the present technique can be in the form of tablet, powder, capsule, syrup, oral gel, or liquid diet.

Acetic acid in the present technique is a chemical represented by a chemical formula CH₃COOH. In addition, the “salt” in an acetic acid salt in the present technique is desirably, but not particularly limited to, a component acceptable in pharmaceuticals, food or drink products, or feeds. Since the acidic taste and acidic odor of acetic acid can be reduced in a salt, the form of salt is preferred.

Acetic acid and/or an acetic acid salt in the present technique may be a fermentation-based one or a chemically-synthesized one, and may be used in a state of liquid (specifically, crude liquid, diluted liquid, or the like) or solid (specifically, powder, granule, or the like). Note that acetic acid that is released from an acetic acid compound, such as an acetic acid ester derivative, by biodegradation or the like may exhibit the efficacy of the present technique.

Suitable examples of the salts include one or two or more selected from metal salts and amine salts.

Examples of the metal salts include alkali metal salts, alkali earth metal salts, and other metal salts.

Examples of the alkali metal salts include lithium salt, sodium salt, and potassium salt. Examples of the alkali earth metal salts include beryllium salt, magnesium salt, and calcium salt. Examples of other metal salts include metal salts, such as copper(II) salt, iron(II) salt, and zinc salt.

Examples of the amine salts include ammonium salt, a basic amino acid salt (for example, lysine salt, arginine salt, and histidine salt), N,N′-dibenzylethylenediamine salt, choline salt, diethanol amine salt, ethylenediamine salt, and trishydroxymethylaminomethane salt.

The salts may be used alone or in combination of two or more thereof.

Acetic acid and/or an acetic acid salt in the present technique may be produced by fermentation or chemical synthesis.

Examples of commercial products of acetic acid and/or acetic acid salts include a vinegar, acetic acid (also referred to as glacial acetic acid), sodium acetate, calcium acetate, ammonium acetate, lithium acetate, magnesium acetate, and zinc acetate. A mixture of acetic acid and an acetic acid salt may be used, and an example is a mixture of sodium acetate and glacial acetic acid. The commercial products may be used alone or in combination of two or more thereof.

Among them, a product that can be orally taken and can be industry mass-produced is preferred in terms of safety and cost and examples thereof include acetic acid metal salts.

As described later in Examples, acetic acid and/or an acetic acid salt in the present technique has an action to suppress a cognitive function decline. Thus, acetic acid and/or an acetic acid salt in the present technique has an action to improve a brain function, brain dysfunction, a cognitive function, cognitive dysfunction, vascular dementia, Alzheimer-type dementia, or the like, and has an action of prophylaxis or treatment of the various symptoms (for example, a brain function decline, brain dysfunction, a cognitive function decline, cognitive dysfunction, vascular dementia, and Alzheimer's-type dementia).

Acetic acid and/or an acetic acid salt in the present technique can be used as an active ingredient of a composition for improving brain function, a composition for brain function decline or dysfunction improvement, a composition for cognitive dysfunction improvement, a composition for cognitive function decline suppression, a composition for vascular dementia improvement, and a composition for Alzheimer's-type dementia improvement. The compositions may be used as a preparation, a food or drink composition, and as a feed.

Acetic acid and/or an acetic acid salt in the present technique can be contained as an active ingredient of various such compositions or preparations. In addition, acetic acid and/or an acetic acid salt in the present technique can be used as an active ingredient for a prophylaxis method, improvement method, and/or therapeutic method related to and for a brain function decline, brain dysfunction, a cognitive function decline, cognitive dysfunction, vascular dementia, and Alzheimer's-type dementia.

Acetic acid and/or an acetic acid salt in the present technique can suppress a decline in a brain function, such as a memory ability or a learning ability and is safe even in the use for a long period of time, and thus can be used as a content of a non-therapeutic food or drink product for prophylaxis or improvement of an age-related cognitive function decline, senility, forgetfulness, or the like.

Then, acetic acid and/or an acetic acid salt in the present technique can be used for prophylaxis, improvement, and/or treatment of a brain function decline or the like as described above as an active ingredient of a pharmaceutical, a quasi-drug, a skin external agent, a pharmaceutical composition, a food or drink product, a food or drink composition, or the like for a human or an animal. The composition of the present invention may be further incorporated into such a preparation or composition. In addition, acetic acid and/or an acetic acid salt in the present technique can be used for producing various such preparations or compositions.

In the present technique, in addition to acetic acid and/or an acetic acid salt in the present technique, an optional component may be used, as needed, in combination therewith. As such an optional component, any component that is acceptable in pharmaceuticals, food or drink products, feeds, and the like may be appropriately used. Examples of such optional components include sugars, sugar alcohols, polysaccharides, pH modifiers, fatty acid esters, corrigents, fragrances, and excipients.

The preparation or the composition of the present technique can be produced by a known production method. For example, since acetic acid and/or an acetic acid salt in the present technique is easily available in the form of powder or liquid, the preparation or the composition of the present technique can be obtained by mixing acetic acid and/or an acetic acid salt with another raw material component (solid, semi-solid, liquid, etc.) or by impregnating another solid raw material component with acetic acid and/or an acetic acid salt.

The content or the amount used in the present technique is not particularly limited and can be appropriately selected according to the gender, the interval between intakes, or the like.

The content in the present technique is preferably 0.001 to 30% by mass in terms of acetic acid, more preferably 0.005 to 10% by mass, further preferably 0.01 to 6% by mass, and furthermore preferably 0.05 to 5% by mass. From the viewpoint of the efficacy and the easiness of oral intake, the content is preferably 0.01 to 2% by mass, more preferably 0.1 to 1.5% by mass, and further preferably 0.5 to 1% by mass.

A standard amount used in the present technique is preferably 2.5 to 500 mg/kg body weight/day in terms of acetic acid, more preferably 12.5 to 250 mg/kg body weight/day, and further preferably 25 to 150 mg/kg body weight/day.

The interval of administrations or intakes in the present technique is not particularly limited, and the administration or intake may be performed once a day or may be performed by several divided portions.

The present technique is desirably administered or taken for at least 5 days, and more desirably for at least 10 days. In addition, the present technique is desirably administered or taken in a continuous daily manner.

The subject of the present technique is not particularly limited but is desirably a middle-aged or older person in which a decline in a brain function is likely to occur, and more preferably an old person. Middle age refers to approximately the late 30's to early 50's, and old age refers to the late 50's or older. In addition, the present technique is desirably administered prior to the onset of dementia.

The manner of administration of the present technique is not particularly limited but examples include oral administration or oral intake, nasal administration, sublingual administration, instillation administration, inhalation administration, transrectal administration, injection (intravenous administration, intramuscular administration, subcutaneous administration), and percutaneous administration. Among them, oral administration or oral intake is more preferred since the compound of the present technique is highly safe and such an administration method is simple and convenient.

When acetic acid and/or an acetic acid salt in the present technique is used in a pharmaceutical, the pharmaceutical may be orally or parenterally administered, and oral administration and an administration manner acting on a mucosa are preferred. Examples of parenteral administrations include injection (blood, skin, muscle, etc.), intrarectal administration, and inhalation. Examples of dosage forms for oral administration include tablet, capsule, troche, syrup, granule, powder, and ointment.

In addition, in formulation into a preparation, in addition to acetic acid and/or an acetic acid salt, components ordinarily used in formulation into a preparation, such as an excipient, a pH modifier, a colorant, and a flavoring agent, can be used. Furthermore, a component having an effect of prophylaxis or treatment of a known or possible future disease can be used together according to the purpose.

Furthermore, according to the administration method, the composition may be appropriately formulated into a desired dosage form. For example, in cases of oral administration, the composition can be formulated into solid preparations, such as a powder, granules, tablets, and capsules; and liquid preparations, such as a solution, a syrup, a suspension, and an emulsion. In cases of parenteral administration, the composition can be formulated into suppositories, a spray, an ointment, patches, and an injection.

In addition, the formulation into a preparation can be appropriately achieved by a known method according to the dosage form. In formulation into a preparation, a fraction from separation or purification thereof alone may be formulated into a preparation, or may be formulated into a preparation with a preparation carrier appropriately incorporated.

As the preparation carrier, according to the dosage form, various organic or inorganic carriers can be used. In cases of solid preparations, examples of such carriers include an excipient, a binder, a disintegrator, a lubricant, a stabilizer, and a corrigent.

Examples of excipients include sugar derivatives, such as lactose, sucrose, glucose, mannitol, and sorbitol; starch derivatives, such as corn starch, potato starch, α-starch, dextrin, and carboxymethyl starch; cellulose derivatives, such as crystalline cellulose, hydroxypropylcellulose, hydroxypropylmethylcellulose, carboxymethylcellulose, and carboxymethylcellulose calcium; Arabic rubber; dextran; pullulan; silicate derivatives, such as light anhydrous silicic acid, synthetic aluminum silicate, and magnesium aluminometasilicate; a phosphate derivative, such as calcium phosphate; a carbonate derivative, such as calcium carbonate; and a sulfate derivative, such as calcium sulfate.

Examples of binders include: in addition to the above excipients, gelatin; polyvinylpyrrolidone; and macrogol.

Examples of disintegrators include, in addition to the above excipients, chemically-modified starch or cellulose derivatives, such as croscarmellose sodium, carboxymethylstarch sodium, and crosslinked polyvinyl pyrrolidone.

Examples of lubricants include: talc; stearic acid; metal stearates, such as calcium stearate and magnesium stearate; colloidal silica; waxes, such as veecum and spermaceti; boric acid; glycols; carboxylic acids, such as fumaric acid and adipic acid; a sodium carboxylate, such as sodium benzoate; a sulfuric acid salt, such as sodium sulfate; Leucine; lauryl sulfate salts, such as sodium lauryl sulfate and magnesium lauryl sulfate; silicic acid compounds, such as anhydrous silicic acid and silicic acid hydrate; and starch derivatives.

Examples of stabilizers include: p-oxybenzoic acid esters, such as methylparaben and propylparaben; alcohols, such as chlorobutanol, benzyl alcohol, and phenylethyl alcohol; benzalkonium chloride; acetic anhydride; and sorbic acid.

Examples of corrigents include a sweetener, an acidulant, and a fragrance.

Note that examples of carriers used in the case of liquid preparations for oral administration include a solvent, such as water, and a corrigent.

When acetic acid and/or an acetic acid salt in the present technique is used as a food or drink product for a human or an animal, acetic acid and/or an acetic acid salt can be added to a known food or drink product or can be mixed in raw materials of a food or drink product to produce a new food or drink product. It is also possible to further incorporate acetic acid and/or an acetic acid salt in the present technique into raw materials of an ordinary food or drink product to thereby additionally achieve the efficacy of the present technique.

The food or drink product may be in any form of liquid, paste, solid, and powder, and examples thereof include tablet confections, liquid diets, feeds (including feeds for pets), wheat flour products, ready-to-eat foods, processed agricultural products, processed marine products, processed livestock products, milk or dairy products, oils or fats, basic seasonings, combined seasonings or foods, frozen foods, confections, drinks, and other commercial products.

Examples of wheat flour products include bread, macaroni, spaghetti, noodles, cake mixes, powder for deep-fried foods, and bread crumbs.

Examples of ready-to eat foods include ready-to-eat noodles, cup noodles, retort or prepared foods, prepared canned foods, foods for microwave cooking, ready-to-eat soups or stews, ready-to-eat miso soups or clear soups, canned soups, freeze-dried foods, and other ready-to-eat foods.

Examples of processed agricultural products include canned agricultural products, canned fruits, jams or marmalades, pickles, cooked beans, dried agricultural products, and cereals (processed grains).

Examples of processed marine products include canned marine products, fish meat hams or sausages, marine paste products, marine delicacies, and foods boiled in soy.

Examples of processed livestock products include canned or paste livestock products and livestock meat hams or sausages.

Examples of milk or dairy products include processed milks, milk beverages, yogurts, lactic acid bacteria beverages, cheeses, ice creams, modified dry milks, creams, and other dairy products.

Examples of oils or fats include butter, margarines, and vegetable oils.

Examples of basic seasonings include soy sauce, Miso, sauces, processed tomato seasonings, Mirin seasonings, and vinegars, and examples of combined seasonings or foods include seasoning mixes, curry rouxes, dipping sources, dressings, noodle soups, spices, and other combined seasonings.

Examples of frozen foods include frozen material foods, frozen semi-prepared foods, and frozen prepared foods.

Examples of confections include caramels, candies, chewing gums, chocolates, cookies, biscuits, cakes, pies, snacks, crackers, Japanese confections, rice confections, bean confections, dessert confections, and other confections.

Examples of beverages include carbonated beverages, natural fruit juices, fruit beverages, refreshing beverages with fruit juice, fruit beverages with pulp, fruit beverage with granules, vegetable beverages, soy milk, soy milk beverages, coffee beverages, tea beverages, powder beverages, concentrated beverages, sport drinks, fortified drinks, alcohol drinks, vinegar-containing beverages, and other favorite drinks.

Examples of other commercial foods than the above include baby foods, Furikake (Japanese rice condiments), and Othazuke Nori (Japanese green tea rice seasonings).

The food or drink product defined in the present technique can be provided or sold as a food or drink product with an indication of a health use.

The “indication” actions include all actions for informing users of the application, including any expression that can evoke or bring to mind that the application falls into the “indication” action of the present technique regardless of the object, content, subject, and medium of the indication.

In addition, the “indication” is preferably provided in such an expression that users can directly recognize the application. Specific examples of indications include: an action to deliver, pass, exhibit for delivery or passing, or import a commodity with respect to a food or drink product with the application written thereon or on a package thereof; and an action to exhibit or distribute advertising, a price list, or a transaction document of a commodity with the application written thereon, or to provide information of such an advertising, a price list, or a transaction document with the application included therein by an electromagnetical method (the Internet or the like).

On the other hand, regarding the content of the indication, the indication is preferably an indication approved by the government or the like (for example, an indication or the like approved under various institutions established by the government and put in a manner based on the approval). The content of the indication is preferably put on a package, a container, a catalog, a pamphlet, an advertising material in the selling site, such as POP, or other documents.

Examples of “indications” also include indications that the commodity is a health food, a functional food, an enteral nutritive food, a food for special uses, a health functional food, a special health food, a nutritive functional food, a functionality-indicated food, a quasi-drug, or the like. Particularly among them, indications approved by Consumer Affairs Agency, for example, indications approved by the institution about special health food or institutions similar thereto are mentioned. Specific examples of the latter indications include an indication that the commodity is a special health food, an indication that the commodity is a conditional special health food, an indication that the commodity may influence on the body structure or function, and an indication about reduction of a disease risk. More specifically, typical examples include an indication that the commodity is a special health food provided in Enforcement Regulations of the Health Promotion Act (Ordinance of the Japanese Ministry of Health, Labor and Welfare No. 86 of Apr. 30, 2003) (especially an indication of a health application) and similar indications thereto.

When acetic acid and/or an acetic acid salt in the present technique is used in a feed, the acetic acid and/or an acetic acid salt may be added to a known feed or may be mixed in raw materials of a feed to produce a new feed.

Examples of raw materials of the feed include grains, such as corn, wheat, barley, and rye; brans, such as wheat bran, barley bran, rice bran, and defatted rice bran; product lees, such as corn gluten meal and corn jam meal; animal-originated feeds, such as skim milk, whey, fish powder, and bone powder; a yeast, such as beer yeast; mineral feeds, such as calcium phosphate and calcium carbonate; oils or fats; amino acids; and sugars. Examples of forms of the feed include feeds for pets (pet foods), livestock feeds, and fish-breeding feeds.

As described above, the present technique can be used in a wide variety of fields of food or drink products, food or drug compositions, functional foods, pharmaceuticals, feeds, and the like.

In the present technique, the following configuration can be used.

[1] A composition for improving brain function containing acetic acid and/or an acetic acid salt as an active ingredient.

[2] The composition for improving brain function according to [1], wherein the brain function improvement is brain dysfunction improvement.

[3] The composition for improving brain function according to [1] or [2], wherein the brain function improvement is cognitive dysfunction improvement.

[4] The composition for improving brain function according to [3], wherein the cognitive dysfunction is Alzheimer's-type dementia or vascular dementia.

[5] The composition for improving brain function according to any one of [1] to [4], which is for oral intake.

[6] The composition according to any one of [1] to [5], wherein the composition is a brain function improver, a food or drink composition for improving brain function, or a pharmaceutical composition for improving brain function.

[7] Use of a food or drug composition containing acetic acid and/or an acetic acid salt in prevention or improvement of forgetfulness or senility for a non-therapeutic purpose.

[8] A method of prophylaxis, treatment, or improvement of a disease or a symptom related to a brain function decline, the method including administering or taking acetic acid and/or an acetic acid salt as an active ingredient or using acetic acid and/or an acetic acid salt.

[9] Acetic acid and/or an acetic acid salt for improving a brain function.

[10] Use of acetic acid and/or an acetic acid salt for producing a composition for improving brain function.

[11] In any one of [8] to [10], the brain function improvement is brain dysfunction suppression.

[12] In any one of [8] to [10], the brain function improvement is cognitive dysfunction improvement.

[13] The cognitive dysfunction improvement according to [12] is prophylaxis, improvement, or treatment of Alzheimer's-type dementia or vascular dementia.

### EXAMPLES

The present technique will be described in more detail below with reference to examples and the like. Note that the examples described below show a typical example of the present technique and the scope of the present technique is not to be narrowly construed by the example.

### Example 1

As an Alzheimer's disease model mouse, a mouse with excess amyloid β administered in the brain ventricle has been widely used (NPL 6: Takeda et al., Brain Research, 1280, 137-147, 2009.). With reference to NPL 6, Alzheimer's disease model mice were prepared according to the following procedure and the improvement effect of acetic acid was evaluated.

A solution containing 150 mM of sodium acetate was administered with drinking water. Taking the day when the administration was started as Day 1, amyloid was injected on Day 3. Specifically, an animal was anesthetized by intraperitoneally administering 40 mg/kg of pentobarbital sodium (Tokyo Chemical Industry Co., Ltd.) (amount of liquid administered: 10 mL/kg). After anesthesia, levobupivacaine hydrochloride (Popscaine (registered tradename) 0.25% inj., Maruishi Pharmaceutical Co. Ltd.) was subcutaneously administered (0.1 mL) to the scalp. After anesthesia, hair of a parietal region of the animal was cut and the head was fixed on a brain stereotaxic apparatus.

The scalp was disinfected with ethanol for disinfection and then was cut to expose the cranial bone.

Using a dental drill, a hole for inserting a stainless steel pipe was made in the cranial bone at 1 mm on the lateral side (right) and 0.2 mm on the rear side of bregma, and a stainless steel pipe with an outer diameter of 0.5 mm connected to a silicone tube and a microsyringe was vertically inserted to a depth of 2.5 mm from the bone surface. Into the brain ventricle, 3 μL of an amyloid β solution (200 pmol/3 μL) was injected with a microsyringe pump over 3 minutes. In a pseudo-operation group, 3 μL of PBS was injected with a microsyringe pump over 3 minutes.

After injection, the head was left to stand for 3 minutes with the stainless steel pipe still inserted and the stainless steel pipe was then slowly detached. Then, the stainless steel pipe was removed and the cranial hole was closed with a non-absorbable marrow hemostatic agent (Nestop (registered tradename), Alfresa Pharma Cooperation), and the scalp was sutured.

The administration of acetic acid with drinking water was continued until the end of the action test.

10 ddY male mice were used for each group.

The measurement method of a cognitive function is as follows.

Y-Maze test: a Y-shaped plastic maze (UNICOM Corporation) composed of three arms each having a length of 39.5 cm, a floor width of 4.5 cm, and a wall height of cm and each branching at 120 degree with respect to the other arms was used in the test. After placing the apparatus, the illumination for the floor of the apparatus was adjusted to 20 Lux.

An animal was placed in any one arm of the Y-shaped maze and was allowed to freely sleuth in the maze for 8 minutes. The order of the arms that the animal moved during the measurement period was recorded and the number of the movements to arms was counted and taken as a total entry number.

Next, the number of combinations in each of which different three arms were continuously selected among the total entry number was checked and taken as a spontaneous alternation action number.

A spontaneous alternation action rate was calculated with the following formula.

Spontaneous alternation action rate (%)=[spontaneous alternation action number/(total entry number−2)]×100

The result is shown in Table 1. It was demonstrated that intake of 150 mM sodium acetate with drinking water improved a cognitive function of Alzheimer disease model mice.

TABLE 1

| | | Spontaneous | |
|---|---|---|---|
| | Administration | alternation | |
| | subject | action rate (%) | S.E. |
| Pseud operation group | Physiological saline | 78.8 | 4.6** |
| Amyloid β injection group | Physiological saline | 53.4 | 2.2 |
| Amyloid β injection group | 150 mM Sodium | 67.5 | 2** |
| | acetate | | |

**p < 0.01 as compared with (Amyloid β + physiological saline) group

1. A composition for improving brain function comprising acetic acid and/or an acetic acid salt as an active ingredient.

2. The composition according to claim 1, wherein the brain function improvement is improvement in brain dysfunction.

3. The composition according to claim 1, wherein the brain function improvement is improvement in cognitive dysfunction.

4. The composition according to claim 3, wherein the cognitive dysfunction is Alzheimer-type dementia or vascular dementia.

5. The composition according to claim 1, wherein the composition is for oral intake.

6. The composition according to claim 1, wherein the composition is a food or drink composition for improving brain function.

7. A method of prophylaxis, treatment, or improvement of a disease or a symptom related to a brain function decline, the method comprising administering or taking acetic acid and/or an acetic acid salt as an active ingredient.

8. Acetic acid and/or an acetic acid salt for improving a brain function.

9. A process for producing a composition for improving brain function, the process comprising:
adding acetic acid and/or an acetic acid salt according to claim 1 to a composition.
